(19) United States
(12) Patent Application Publication
(10) Pub. No.: US 2006/0153792 A1
(43) Pub. Date: Jul. 13, 2006

(54) Novel cosmetic formulations based on a gel-forming and/or thickening agent and applications of same

(76) Inventors: Jean-Pierre Arnaud, Thiais (FR); Agnes Le Fur, Nogent-Sur-Marne (FR)

Correspondence Address: HEDMAN & COSTIGAN P.C., 1185 AVENUE OF THE AMERICAS, NEW YORK, NY, 10036, US

(21) Appl. No.: 10/530,082

(22) PCT Filed: Oct. 2, 2003
(86) PCT No.: PCT/FR03/02898

§ 371 Date: Dec. 8, 2005

(30) Foreign Application Priority Data
Oct. 2, 2002 (FR) 02/12202

(51) Int. Cl. A61K 8/81

(52) U.S. Cl. 424/70.16

(57) ABSTRACT

The invention relates to the field of the necessities of life and more particularly to the field of cosmetics, hygiene and skin products. More specifically it relates to aqueous, fluid or gelled, cosmetic compositions intended to be applied to the skin, the mucous membranes or the teguments, characterized in that they contain, as active ingredient, a gelling and/or thickening agent formed from a combination of three constituents, namely a polyacrylamide and ammonium acrylate copolymer and/or an anionic acrylic copolymer, lecithins or phospholipids of plant origin, and a polyglyceryl acylate, in combination or in a mixture with excipients or appropriate vehicles for topical use. These compositions can incorporate other appropriate active ingredients in particular for use in cosmetics. Use in cosmetics or in skin products for the production of perfectly stable emulsions, gels, or creams.

The present invention relates to the field of the necessities of life and more particularly to the field of cosmetics, hygiene and skin products.

A more precise subject of the invention is novel compositions intended for skin care, the feature of which is to form a gel or to produce an emulsion with or without the use of an additional surfactant.

A more specific subject is aqueous, fluid or gelled compositions intended to be applied to the skin, the mucous membranes or the teguments, characterized in that they contain, as active ingredient, a gelling and/or thickening agent formed from a combination of three constituents: a polyacrylamide and ammonium acrylate copolymer and/or an anionic acrylic copolymer, phospholipids or lecithins of plant origin, and a polyglyceryl acylate, in combination or in a mixture with excipients or vehicles which are more particularly appropriate for topical use.

The compositions according to the invention have significant advantages in comparison to those already produced according to the prior art which contain, as gelling agent, derivatives of cellulose or vegetable gums. In fact, these novel compositions have on the one hand a soft and agreeable feel and on the other hand, show no risk of alteration. This is shown by the fact that they have no tendency for the chemically-active substances that they contain to recrystallize, in particular in the event of a variation in temperature or when the formulations contain active ingredients which are not very soluble, having a tendency to recrystallize. This stability is in particular shown by the study of the viscosity as a function of parameters such as the operating method, pH, alcohol, electrolytes, raw materials, and presented in Examples V and VI).

The compositions incorporating the gelling and/or thickening agent according to the invention have, in particular, the advantage of being perfectly stable over a wide range of temperatures and being able to be preserved without risk of recrystallization or surface desiccation if the containers are stored open or incompletely resealed.

Moreover, the gelling and/or thickening agent according to the invention can constitute an emulsifying agent as it allows emulsions to be produced either cold or hot, without an additional surfactant-type emulsifier, and without the need to resort to a calculation of the Hydrophile/Lipophile Balance (HLB) coefficient. Therefore in a particularly surprising manner, the emulsifying agent according to the invention can be added to the oil phase before carrying out the emulsifying phase by adding an aqueous vehicle. Example IV therefore shows that it is possible to emulsify up to 40% of oil in a composition according to the invention containing 3% by weight of gelling and/or thickening agent.

Moreover, the agent according to the invention can constitute a thickening and stabilizing agent as it can be added hot to an oil-in-water emulsion and thus allows thickening and stabilizing, in particular during cooling down. This ability has been demonstrated in particular in the study of the influence of the temperature of the formulation process on the viscosity of the solution and presented in Example VI) B).

The agent according to the invention can also constitute a good gelling agent. Moreover, it confers remarkable properties on the compositions which incorporate it. Experiments, the results of which are presented in Example III, have demonstrated that such a gelling and/or thickening agent generates, on application of the fluid or gelled composition, a freshening effect which is much better than that observed with usual gelling agents. An increase of the order of 9.4% has in fact been observed. Moreover, the stickiness which often constitutes a drawback observed with conventional gelling agents is in this case attenuated to a very remarkable extent, by almost 17.5%.

The gelling and/or thickening agent according to the invention retains its properties over a wide pH range, being able to range from 2 to 12, and is compatible with ethanol up to a maximum percentage of 40% of the total weight of the composition. Moreover, it offers a good resistance to electrolytes such as salts in general, but also to certain substances which are particularly difficult to formulate such as magnesium ascorbyl phosphate used as an active depigmenting agent and often incompatible with a good number of conventional gelling agents. This stability was the subject of a study presented in particular in Examples V and VI. The compositions which incorporate this gelling and/or thickening agent are thus particularly shell stabilized.

All these remarkable properties thus make this gelling and/or thickening agent a particularly useful compound for the formulation of cosmetic, pharmaceutical and in particular dermatological compositions or also for other industrial purposes (detergent, paper, building, construction, petrochemical).

The important element of the invention resides in the use of a gelling and/or thickening agent which, according to the invention, is based on phospholipids or lecithins, of plant origin. The latter, in fact, in particular contribute to the excellence of the properties of the gelling and/or thickening agent according to the invention.

According to the invention, the phospholipids of plant origin and the lecithins are in particular those extracted from of soybean oil, rice oil or sunflower oil. Phospholipids derived from other oleaginous plants or oils such as rapeseed, poppyseed, corn, babassu, thistle oil, Limnanthes alba (prairie grass) oil, milkweed seed oil or wheat germ oil can also be used. The preferred phospholipids according to the invention are those derived from sunflower oil. Those extracted from soybean, rapeseed, rice, oats are also compatible with the invention. The phospholipids, in particular those extracted from sunflower seed, are known substances, and are already commercially available, most often in the form of a transparent liquid with an amber-brown colour, which spreads as a fine clear film. This is a standardized product the viscosity of which is constant. This product contains no raw material originating from soya or wheat. Consequently it is not subjected to any regulations concerning genetically modified organisms, in accordance with EEC regulations 49/2000 and 50/2000.

The addition of these sunflower phospholipids has already been described in the food industry and in particular in the production of chocolate paste, (Russian patent 2058743 in the name of Krasd Poly) as improving the quality and reducing the viscosity of the mixture of ingredients. These phospholipids moreover reduce the tendency of the fats to crystallize and inhibit rancidity.

It is also noted that the addition of water to a gel notably increases its viscosity but conversely that the addition of sunflower phospholipids notably reduces this drawback.

The sunflower phospholipids also have the advantage of not degrading with storage and retain their properties intact above 15° C., however without the temperature exceeding 60° C.

The gelling and/or thickening agent according to the present invention is formed from the combination of at least three constituents: a polyacrylamide and/or ammonium acrylate copolymer and/or an anionic acrylic copolymer, phospholipids or lecithins of plant origin, and a polyglyceryl acylate, optionally in combination or in a mixture with excipients or vehicles appropriate for cosmetic or dermatological use.

By the term “pbospholipids”, is meant according to the invention glycerides containing an orthophosphoric acid radical, linked by an ester function.

By the term “lecithin”, is meant according to the invention a mixture of at least 60% of phospholipids in oil in particular sunflower oil or sunflower triglycerides.

According to the invention, the acrylic copolymer can be in particular a sodium acrylate/acryloyldimethyl taurate copolymer. This copolymer, which is particularly useful according to the invention, is marketed in a dilute form called Flocare DP/ET36 LM (Company SNF SA) which constitutes a liquid dispersion of the polymer at 60% by weight in hydrogenated polydecene at 40% and in a trideceth-6 at 10%. This product is in particular described in the patent WO01/97772 in the name of SNF SA.

The polyacrylamide and ammonium acrylate copolymer and the anionic acrylic copolymer constitute products which are commercially available.

According to the invention, the polyglyceryl acylate is preferably a stearate, a distearate, a palmitate or a polyglyceryl linoleate. Preferably, the polyglyceryl acylate is a polyglyceryl-10 acylate. The polyglyceryl acylate, in particular polyglyceryl stearate and in particular polyglyceryl-10 stearate is a known product and is already commercially available.

In the gelling and/or thickening agent according to the invention, the respective proportions of each of the constituents can range within the following wide limits, expressed as a percentage of the total weight of the agent:

from 20 to 50% copolymer and preferably from 30% to 50%

from 6 to 40% phospholipids and preferably from 20% to 30%

from 5 to 40% polyglyceryl acylate and preferably from 10% to 30%

For reasons of convenience, the preferred gelling and/or thickening agent contains equal quantities of copolymers, phospholipids of plant origin and polyglyceryl acylate in a mixture. The gelling and/or thickening agent can moreover only be constituted by these three components thus completing the agent to 100% by weight.

In this form, the gelling and/or thickening agent can be incorporated directly into the compositions. It can also be combined in a physical combination containing pure dilute oil or a polydiene or an isononyl ester. Preferably, it is combined with a polymer carrier such as a hydrogenated polyisobutene.

A subject of the invention is therefore also the gelling and/or thickening agent formed from three constituents polyacrylamide and/or ammonium acrylate copolymer and/or anionic acrylic copolymer, phospholipids of plant origin and glyceryl acylate combined in a physical combination containing pure oil or a polydiene or an isononyl ester.

It should be noted that the concentrations are given as percentages of active ingredients.

The polymer can thus be presented in dilute form, in particular in polydiene and, in this case, a quantity ranging from 50 to 80% of copolymer in dilute form will be used, said dilute form containing from 40 to 60% of copolymer active ingredient.

The phospholipids can be presented in the form of lecithins and in this case, a quantity of lecithin ranging from 10% to 40% of the total weight of the agent is used. In this case, the lecithins according to the invention containing at most 40% of oil in the form of triglycerides, the gelling and/or thickening agent will contain a concentration of oil, in particular of sunflower oil less than 16% of the total weight of the agent.

The present invention makes available a novel type of gelling and/or thickening agent presented in the form of powder or gel, formed of non-toxic substances. The agent, in particular when it exclusively contains a polymer, phospholipids in oil and a glyceryl acylate, can be in solid form, and it is then possible to reduce it to powder by pulverization.

However, for better handling and homogenization, the agent according to the invention will preferentially be used in gelled form. It is then presented in the form of a fluid orange-coloured gel, which can be urged over a wide pH range from 2 to 12, preferably from 4 to 8, preferentially at an optimum pH from 5 to 7. The viscosity in water of a 2% suspension of the agent according to the invention is then of the order of 75,000 cPs, determined with an LGV6 Rheoviscosimeter. The viscosity of a 1 to 5% aqueous gel of is comprised approximately between 9,000 cPs and 400,000 cPs. A study the results of which are presented in Example VI) A) has shown that the gelling power of the agent according to the invention can be observed very clearly from a concentration of 0.5% and is shown to be very significant from 3%.

In fact, a subject of the invention is also the fluid or gelled aqueous compositions characterized in that they contain as active ingredient at least one gelling and/or thickening agent according to the invention, in combination or in a mixture with excipients or vehicles appropriate for use, in particular cosmetic or dermatological use.

The fluid or gelled compositions according to the invention generally contain a concentration which ranges from 0.05% to 10% and preferentially from 0.2 to 4% of gelling and/or thickening agent defined previously relative to the total weight of the composition.

In fact, it was noted that it was possible, with this gelling and/or thickening agent, to produce cosmetic compositions in the form of gels which can incorporate one or more active ingredients or principles. The gelled compositions according to the invention contain in particular a concentration which ranges from 0.10 to 10% of the gelling and/or thickening agent and preferably from 0.2 to 5%. Preferably, the most advantageous composition contains from 0.5 to 5% of agent and preferentially from 0.5 to 3%.

The gelling and/or thickening agent according to the invention can moreover serve as emulsifying agents or vectors for a large number of cosmetic preparations. A composition containing 3% by weight of the gelling and/or thickening agent (Example III) has therefore allowed the emulsification of up to 40% of oil. In the oil-in water emulsions, the addition of the gel according to the invention, at a dose ranging from 0.20 to 10%, preferentially from 0.2 to 7.5%, and very advantageously from 0.2 to 4% in the oil phase until complete hot or cold dispersion, therefore allows the preparation of fluid or viscous emulsions, with perfect stability.

The compositions according to the invention, in particular those which are celled, can contain a very variable proportion of water which allows the viscosity of the preparation to be adjusted. In general it ranges between 5 and 90% by weight and in particular between 20 and 70%. Such a content confers on the gel a fine texture and a refreshing sensation. Therefore, the application of the gels according to the invention produces an impression of freshness which is not due to the evaporation of a solvent or to the volatilization of a product with high vapour pressure. For the production of the O/W-type emulsion according to the invention, 30 to 70% water is preferably used.

Preferentially, the compositions according to the invention are used in the form of emulsion, and in particular oil-in-water emulsion. Preferably, by the term emulsion is meant a standard dispersion. The grain size distribution study presented in Example 7 showed that, in oil-in-water-type emulsions according to the invention, produced at 1000 and 3000 rpm, the average size of the particles is mostly comprised between 1 μm and 20 μm, or even between 1 μm and 11 μm.

Apart from the hot or cold formulation facilities of the emulsions previously disclosed, the gelling and/or thickening agent has the surprising advantage of being able to be incorporated both into the oil phase and into the aqueous phase in order to produce an oil-in-water or water-in-oil-type emulsion.

No other additional surfactant is necessary and the compositions according to the invention can contain only the gelling and/or thickening agent according to the invention. The compositions according to the invention can, moreover, incorporate one or more active principles or ingredients, in particular those selected from the anti-bacterial agents, viscosity agents, plasticizing agents, hydrating agents, phytoestrogens, amino acids, α-hydroxylated acids, silicones, fatty acid derivatives, texture products, self-tanning agents, organic or mineral anti-sun filters, pigmentation agents, depigmenting agents, sea water, anti-oxidants, vitamins, slimming agents.

It is also possible to incorporate into these gelled or fluid compositions according to the invention, ceramides or pseudo-ceramides, triglycerides of saturated or non-saturated fatty acids, plasmalogens, other lipids such as those extracted from gluten, tri-saccharides, neutral lipids, glycolipids and other phospholipids. They can also incorporate flavouring or perfuming agents, colorants, mineral pigments, such as iron oxides, filling substances, oily agents such as oils or fats of vegetable origin, fats of animal origin (lanolin, suint), synthetic oils (perhydrosquatene), silicone oils (cyclomethicone), fluorinated oils (perfluorinated polyethers, perfluorodecaline), fatty alcohols (cetyl alcohol), waxes (carnauba wax, montana wax, ozokerite, Perilla wax), other lipophilic gelling agents, modified clays, bentones, metallic salts of fatty acid, hydrophobized silica, polyethylenes, mica or other substances used in cosmetics.

As sun protection agent there can in particular be used, known organic and/or mineral physical filters, such as, for example, micronized particles of metal oxide, in particular titanium, iron, cerium, aluminum oxides, particles of metallic silicates, organic compounds such as methylene bis-benzotriazolyl tetramethylbutylphenol (MBBT marketed by Ciba). For the production of sun-care products, the choice of filter used will be determined according to final consistency of the composition according to the invention.

The compositions according to the invention can also incorporate active ingredients for preparations of the anti-ageing cream, anti-wrinkle cream type, such as make-up foundations, such as infant hygiene cream, lipstick preparations, softening agents, such as jojoba oil esters, other thickening agents such as Lanette wax, agents for making the skin supple, such as hazel nut oil or avocado oil, moisturizing agents such as a Jojoba extract. It is also possible to incorporate into the gels an agent which lightens the complexion and/or attenuates the visible effect of wrinkles such as the calcium and aluminum-borosilicate-based product marketed under the name of LUXIL (so-called “soft-focus” lightening effect).

The gels according to the invention are suitable for producing cosmetic creams in particular with moisturizing, anti-wrinkle, slimming power, with anti-irritant power, for producing products for perfectly tolerated ocular or palpebral use, skin creams with very good cutaneous tolerance and having an unctuous feel, very fresh and ver, emollient, baby creams, creams for treating nappy rash, lip colour in the form of a stick, applicators or a liquid preparation to be applied with a brush. The same preparations can moreover be presented in the form of aerosols, milks or lotions.

It is also possible to incorporate opalescent agents or agents with a mother-of-pearl or sparkling appearance, in particular in oil-in-water emulsions for skin care, for the production of skin protection products, make-up or on the other hand for make-up-removing creams.

A subject of the present invention is therefore the use of the gelling and/or thickening agent predefined as a gelling agent, and/or as a thickening agent, or as an emulsifying agent, or as a stabilizing agent, or as an agent improving the feel and in particular the sensation of freshness to the touch in compositions of an industrial character, in particular cosmetic, pharmaceutical and in particular dermatological compositions, more specifically intended to be applied to human or animal skin, teguments and mucous membranes.

A subject of the invention is also a process for producing oil-in-water emulsion, consisting of incorporating in the oil phase the gelling and/or thickening agent according to the invention preferentially in the form of a gel, and to add an aqueous phase to the latter.

The compositions according to the invention have no toxicity nor local intolerance. Nor are they allergenic.

The following examples are presented in order to illustrate the invention and should in no event be considered as a limit to the scope of the invention. Unless otherwise mentioned, the concentrations are given in percentages with respect to the total weight of the composition.

### EXAMPLE I

### Examples of Gels According to the Invention

In the examples hereafter, the gelling and/or thickening agent according to the invention is used in the form of gel with a particularly interesting composition, according to the present invention and called Lucagel.

The agent Lucagel is composed of approximately;

| | | |
|---|---|---|
| | sodium acrylate/acryloyldimethyl taurate copolymer | 28 to 46% |
| {open oversize parenthesis} | | |
| | hydrogenated polyisobutene | 21 to 34% |
| | phospholipids | 6 to 24% |
| {open oversize parenthesis} | | |
| | sunflower oil | 4 to 16% |
| | polyglyceryl-10 stearate | 10 to 20% |

### EXAMPLE II

### Examples of Uses of the Gelling and/or Thickening Agent

As Aqueous Phase Thickening Agent

The complex gelling product called Lucagel disperses in water under simple stirring, without previous swelling and without requiring the addition of a neutralizing agent.

The viscosity of the aqueous gel containing from 1 to 3% of active ingredient ranges from 14,000 to 100,000 cPs. The pH of such a gelled product ranges from 5.5 to 8.5. The gel is sensitive to electrolytes from a concentration of 0.025%. It is compatible with ethanol up to a concentration of 30%.

In practice, gels are produced containing 2 to 3% Lucagel which is dispersed in an aqueous phase or in an oily phase. The gelling can also be introduced after emulsification within the framework of an emulsion stabilization. The dispersion can be carried out cold or hot without difficulty.

For the Production of Oil-in-Water Emulsions

Operating method 1: 2 to 3% Lucagel is dispersed in the lipid phase of an oil-in-water emulsion, as well as mineral, vegetable oils, siliconized fatter acid esters, chemical derivatives of cholesterol. It is also possible to use mixtures of lipid products. They are incorporated cold or hot in the oil phase. The emulsion is then produced by adding an aqueous phase, until complete dispersion, with or without the addition of additional surfactant or by adding the oil phase to the aqueous phase under stirring.

Operating method 2: 1 to 3% Lucagel is dispersed in the cold or hot aqueous phase. The emulsion is then produced by adding the oil phase to the aqueous phase or vice versa under stirring.

As Thickening and Stabilizant Agent of an Oil-in-Water Emulsion

An oil-in-water emulsion is produced, by dispersion of an aqueous phase in an oily solution formed from paraffin oil to which cotton oil and sweet almond oil are added. The emulsion is heated to 30° C. During cooling down, the agent in the form of pure gel according to the invention is then added.

It is also possible to incorporate into the emulsion a flower essence or a flavouring product such as irone, ionones, cinnamic aldehyde or phenylacetic acid.

### EXAMPLE III

### Sensory Study of the Effect of the Gelling and/or Thickening Agent in the Compositions Incorporating It

Operating Method:

General Conditions:

In accordance with the standard NF ISO 11036 and in order for the tests to be statistically valid, the tests were carried out on 20 qualified subjects who are expert panellists having followed a “touch” training course and after evaluation of their reproducibility and sensory discrimination abilities.

The tests were carried out under conditions provided by the standard V09-105. Thus, the products were distributed into white-coloured pots, identified by the sensory analysis laboratory organisation's own code. The profile of each product is determined upon spreading then one minute later. Under individual and independent test conditions, each ex-pert tested two samples simultaneously: the product according to the invention and a reference gelling product, comprising polyacrylamide. C13-C14 isoparaffin and laureth-7. The expert then transcribed these differences in sensation relative to the standard using a measurement grid from 0 to 10, the sensory difference being considered as zero at a value of 10.

An average comparison test is then carried out in order to identify the significant differences between the products.

In order to interpret this test, the value t 2.08 was then used corresponding to the value of Student's t table for 70 tests with a 5% risk.

The of confidence interval of each average is calculated (CI) according to the formula:
CI=(standard deviation/number of tests)×(2.08)

Then the differences between the averages are produced for each descriptor according to the following formula:
(Average (higher Influence of the operating method value)−CI)−Average (lowest value)+CI)=X

The difference is said to be significant, when X is strictly greater than 0.

Results and Interpretation:

The freshness effect observed is increased by 9.4% in comparison with the gelling products used up to now.

The stickiness observed is reduced by 17.5% in comparison with the gelling products used up to now.

### EXAMPLE IV

### Maximum Quantity of Emulsifiable Oil

The viscosity of compositions containing purified water is measured (qsf 100%), Phenonip (phenoxyethanol+paraben esters (Clariant), 0.5%), Cetiol SN (Butyrospermum parkii otherwise known as Karite butter (Cognis), qs), and a variable dose of gelling and/or thickening agent. The quantity of emulsifiable oil constitutes the other variable. The following results are obtained:

| Percentage of the agent | Maximal percentage of |
|---|---|
| in the composition | emulsifiable oil |
| 0 | 0 |
| 1 | 15 |
| 2 | 25 |
| 3 | 35 |
| 5 | 40 |

FIG. 1 shows these results in graph form and represents the maximum percentage of emulsifiable oil as a function of the percentage of gelling and/or thickening agent by weight of the composition.

The gelling and/or thickening agent thus has remarkable emulsifying properties, from low concentrations. At concentrations of the order of 3 to 5%, it in fact allows emulsification of up to 40% of the weight of the composition in oil.

### EXAMPLE V

### Study of the Compatibility of the Gelling and/or Thickening Agent with the Raw Materials

The tested composition contains:

| Purified water | qsf 100% | |
|---|---|---|
| Phenonip (phenoxyethanol + paraben esters) | 0.5% | by weight |
| (Clariant) | | |
| Oil | 25% | by weight |
| Agent according to the invention | 3% | by weight |
| Component tested | 3% | by weight |

The viscosity is then measured and the following results are obtained:

| Component tested | Viscosity in cPs |
|---|---|
| Petrolatum | 23,470 |
| Cetearyl isononanoate | 42,250 |
| Prunus amygdalus dulcis (sweet almond oil) | 38,580 |
| C12/C15 alkyl benzoate | 27,560 |
| Ethylhexyl methoxycinnamate + | 31,060 |
| C12/C15 alkyl benzoate (50/50) | |
| Silicone | 15,570 |
| Cetearyl isononanoate + 5% DHA | 28,320 |

It is thus observed that it is possible to use different raw materials, without altering the agent's gelling power. The gelling and/or thickening agent according to the invention is thus compatible with a good number of the standard components of cosmetics, such as in particular the silicones. Prunus extracts (sweet almond), 5% DHA.

### EXAMPLE VI

### Influence of Different Parameters on the Viscosity of the Composition According to the Invention

A. Influence of the Concentration of Gelling and/or Thickening Agent According to the Invention:

The gelling and/or thickening agent is added according to different percentages (0.5, 1, 2, 3, 5) to purified water, the viscosity is then measured and the following results are obtained.

| gelling and/or thickening agent (as a percentage | |
|---|---|
| of the total weight of the composition) | Viscosity in cPs |
| 0 | 0 |
| 0.5 | 1,390 |
| 1 | 9,330 |
| 2 | 46,840 |
| 3 | 99,470 |
| 5 | 400,000 |

FIG. 2 represents the viscosity (in cPs) as a function of the concentration of gelling and/or thickening agent.

It is noted that a gaussian “dose-effect” relationship exists. Thus, the gelling pourer of the agent according to the invention can be observed from a concentration of 0.5%. From 3%, the gelling effect is particularly strong.

B. Influence of the Operating Method:

The formulation tested has the following composition:

| | | |
|---|---|---|
| Purified water | 75% | by weight |
| Phenonip (phenoxyethanol + paraben esters) | 0.5% | by weight |
| (Clariant) | | |
| Cetiol SN | 25% | by weight |
| (Karite butter, Cognis) | | |
| Gelling agent according to the invention | 3% | by weight |

The compositions are produced according to the methods usually used for producing emulsions, phase inversions, and gels in water. The viscosity measurements (in cPs) are as follows:

| Type of composition | | |
|---|---|---|
| produced | Cold process | Hot process |
| Direct emulsion | 11,080 | 9,560 |
| Phase inversion | 14,890 | 10,290 |
| Gel in water | 9,880 | 5,480 |

It is thus observed that cold processes make it possible to obtain a greater viscosity for the same dose of gelling agent. Moreover, the gelling power is only slightly altered by heat and remains very strong. The gelling agent therefore resists heat relatively well and can therefore be used in preparations requiring hot formulation processes.

C. Influence of Electrolytes:

The viscosity of a solution containing different concentrations of sodium chloride is measured:

| | | |
|---|---|---|
| Cetiol SN (karite butter, Cognis) | 25% | by weight |
| Phenonip (phenoxyethanol + paraben esters) | 0.5% | by weight |
| (Clariant) | | |
| Oil | 25% | by weight |
| Gelling agent according to the invention | 3% | by weight |

| | |
|---|---|
| NaCl | variable |
| Purified water | sqf |

The following results are obtained:

| NaCl in % by weight | |
|---|---|
| of the composition | Viscosity in cPs |
| 0 | 52,330 |
| 0.05 | 100,000 |
| 0.1 | 51,980 |
| 0.15 | 43,330 |
| 0.2 | 36,150 |
| 0.3 | 29,220 |
| 0.4 | 22,600 |
| 0.5 | 16,710 |
| 1 | 3,660 |

FIG. 3 represents the viscosity as a function of the concentration of sodium chloride.

It is thus observed that the gelling power is relatively constant for salt concentrations of less than approximately 0.4%. Beyond this, the gelling effect, although still present, is considerably weakened.

Moreover, it is noted that the viscosity is kept high for concentrations comprised between 0 and 0.2% of NaCl. This is particularly observable at an NaCl concentration of approximately 0.1%.

D. Influence of Alcohol:

The viscosity of a solution containing different concentrations of alcohol is measured:

| | | |
|---|---|---|
| Cetiol SN (karite butter, Cognis) | 25% | by weight |
| Phenonip (phenoxyethanol + paraben esters) | 0.5% | by weight |
| (Clariant) | | |
| Gelling agent according to the invention | 3% | by weight |

| | |
|---|---|
| Alcohol | variable |
| Purified water | sqf |

The following results are obtained:

| Alcohol in % relative | |
|---|---|
| to the weight of the | |
| composition | Viscosity in cPs |
| 0 | 52,330 |
| 5 | 53,800 |
| 10 | 38,310 |
| 20 | 27,880 |
| 30 | 11,390 |
| 40 | 0 |

FIG. 4 represents the viscosity as a function of the alcohol concentration.

It is observed that the viscosity is not altered by alcohol concentrations up to 5% by weight relative to the weight of the concentration. The gelling power, although reduced, is also considerable for concentrations of the order of 10% to 20%. Beyond 20%, the gelling power diminishes in a linear fashion and disappears at approximately 40%. This is therefore the alcohol dose limit.

E. Influence of pH:

The viscosity of a solution initially at a pH of 5.68 and adjusted to different pHs is measured:

| | | |
|---|---|---|
| Cetiol SN | 25% | by weight |
| (karite butter, Cognis) | | |
| Phenonip(phenoxyethanol + | 0.5% | by weight |
| paraben esters) (Clariant) | | |
| Gelling agent according to the invention | 3% | by weight |

| | |
|---|---|
| pH adjustment agent | |
| Purified water | sqf |

Citric acid is used as acid adjustment agent and soda as basic adjustment agent.

The following results are obtained:

| pH | Viscosity in cPs |
|---|---|
| 2.47 | 45,520 |
| 3.16 | 46,350 |
| 4.08 | 54,240 |
| 4.87 | 55,000 |
| 5.68 | 54,670 |
| 6.57 | 55,000 |
| 7.42 | 55,000 |
| 8.75 | 46,880 |
| 9.62 | 41,980 |
| 10.81 | 38,320 |
| 12.12 | 31,910 |

FIG. 5 represents the variations in viscosity as a function of the pH.

It is observed that the viscosity is relatively constant over a pH range of 2 to 12, and remains greater than 30.000 cPs for a gelling agent concentration of 3%. In particular it is noted that between the pH values of approximately 4.08 and 7.42, the gelling effect is virtually unaltered.

### EXAMPLE VII

### Granulometry Study of an O/E Emulsion Containing the Gelling and/or Thickening Agent According to the Invention

The composition tested contains as a percentage of the total weight:

| | |
|---|---|
| Purified water | 73.85% |
| Butylene glycol | 3% |
| Glycerin | 2% |
| EDTA, 4Na | 0.05% |

diminishes in a linear fashion and disappears at approximately 40%. This is therefore the alcohol dose limit.

E. Influence of pH:

The viscosity of a solution initially at a pH of 5.68 and adjusted to different pHs is measured:

| | | |
|---|---|---|
| Cetiol SN | 25% | by weight |
| (karite butter, Cognis) | | |
| Phenonip(phenoxyethanol + | 0.5% | by weight |
| paraben esters) (Clariant) | | |
| Gelling agent according to the invention | 3% | by weight |

| | |
|---|---|
| pH adjustment agent | |
| Purified water | sqf |

Citric acid is used as acid adjustment agent and soda as basic adjustment agent.

The following results are obtained:

| pH | Viscosity in cPs |
|---|---|
| 2.47 | 45,520 |
| 3.16 | 46,350 |
| 4.08 | 54,240 |
| 4.87 | 55,000 |
| 5.68 | 54,670 |
| 6.57 | 55,000 |
| 7.42 | 55,000 |
| 8.75 | 46,880 |
| 9.62 | 41,980 |
| 10.81 | 38,320 |
| 12.12 | 31,910 |

FIG. 5 represents the variations in, viscosity as a function of the pH.

It is observed that the viscosity is relatively constant over a pH range of 2 to 12, and remains greater than 30,000 cPs for a gelling agent concentration of 3%. In particular it is noted that between the pH values of approximately 4.08 and 7.42, the gelling effect is virtually unaltered.

### EXAMPLE VII

### Granulometry Study of an O/E Emulsion Containing the Gelling and/or Thickening Agent According to the Invention

The composition tested contains as a percentage of the total weight:

| | |
|---|---|
| Purified water | 73.85% |
| Butylene glycol | 3% |
| Glycerin | 2% |
| EDTA, 4Na | 0.05% |

Operating Method:
1. The two phases A and B are heated to approximately 70-75° C.
2. Phase B is introduced into phase A and this mixture is stirred until a homogeneous emulsion is obtained.
3. During the cooling down of this mixture, phases C, D, E and F are added successively without ceasing to stir.

Rejuvenating Cream:

Composition in Percentages:

| Phase | Ingredient | INCI (Suppliers) | in g |
|---|---|---|---|
| A | Soya isoflavones | Soybean (Soya | 1.50 |
| | | glycine) sterols | |
| | | (Acatris) | |
| | Karite butter | Butyrospermum parkii | 1.00 |
| | | (Cognis) | |
| | Wax Lanette C16 | Cetyl Alcohol (Cognis) | 0.85 |
| | Lanette O | Cetearyl Alcohol (Cognis) | 1.25 |
| | Hazel nut oil | Hazel Nut Oil | 1.50 |
| | | (Alban Muller) | |
| | Luxil | Calcium aluminium | 3.00 |
| | | borosilicate (Potters) | |
| | Germaben II | 30% diazolidinyl urea + | 0.80 |
| | | 11% methyl paraben + | |
| | | 3% propyl paraben + | |
| | | 56% propylene glycol | |
| B | Water | | 75.00 |
| | Ultrez 10 | Carbomer (Noveon) | 0.15 |
| | Lucagel | | 2.00 |
| | Herbasol green tea | Camellia sinensis | 1.00 |
| | | extract (Cosmetochem) | |
| C | 1401 fluid | Dimethicone Copolyol | 0.50 |
| | | (Dow Corning) | |
| D | Blue No. 1 | FDC Blue 1 (LCW) | qs |
| | Serine | Serine (Ajinomoto) | 0.15 |
| | Histidine | Histidine (Ajinomoto) | 0.15 |
| | Arginine | L-Arginine (Ajinomoto) | 0.30 |
| E | Lovin | Fragrance | 0.24 |

Operating Method:
1. The two phases A and B are heated to approximately 70-75° C.
2. Phase A is emulsified in phase B under stirring.
3. During cooling down, phases C, D and E are introduced successively.
4. The final mixture is stirred until a homogeneous cream is obtained.

Anti-Ageing Moisturizing Cream:

In this composition, Lucagel is used as stabilizing agent of another emulsifying agent.

Composition in Percentages:

| Phase | Ingredient | INCI (Suppliers) | in % |
|---|---|---|---|
| A | Demineralized | | qsf 100% |
| | water | | |
| | Satiaxane CX 91 | xanthan gum (Laserson) | 0.20 |
| | EDTA, 4Na | Tetrasodium EDTA(Lambert | 0.02 |
| | | Rivière) | |
| | Glycerin | Glycerin (Interchimie) | 2.00 |
| | Butylene glycol | Butylene Glycol (Interchimie) | 3.00 |
| B | Trimethyl | Betaine (Finfeeds/LMC) | 4.00 |
| | glycine | | |
| | Amisol ® Soft | behenic alcohol + | 4.00 |
| | | glyceryl stearate + | |
| | | soybean sterols (soya glycine) | |
| | | (Lucas Meyer Cosmetics) | |
| | Vitamin E | tocopheryl acetate (Laserson) | 0.50 |
| | acetate | | |
| | Nipanox BHT | BHT (Clariant) | 0.05 |
| | Cetiol SB 45 | butyrospermum parkii (Cognis) | 2.50 |
| | Phytosqualane | Squalane (Sophim) | 5.00 |
| | Tegosoft CO | cetyl octanoate (Degussa) | 3.00 |
| | Eutanol G16S | hexyldecyl stearate (Cognis) | 3.00 |
| | Isopropyl | isopropyl palmitate (Cognis) | 2.00 |
| | palmitate | | |
| | Lanette 16 | cetyl alcohol (Cognis) | 0.60 |
| | Palmitic Add | Palmitic Acid (Dubois) | 0.40 |
| | Abil Wax 2440 | behenoxy dimethicone (Degussa) | 0.50 |
| | Abil 350 | dimethicone (Degussa) | 0.80 |
| | DC 345 | cyclopentasiloxane | 3.00 |
| | | (Dow Corning) | |
| | Parsol MCX | ethylhexyl methoxycinnamate | 5.00 |
| | | (Givaudan) | |
| C | Lucagel | | 0.40 |
| D | Euxyl K400 | Phenoxyethanol + | 0.20 |
| | | methylbromoglutaronitrile | |
| | | (Seppic) | |
| E | Mamaku V. E | Water + | 2.00 |
| | | cyatheaceae extract | |
| | | (Lucas Meyer Cosmetics) | |
| F | C-3810 | Fragrance (Vanessence) | 0.35 |

Operating Method:
1. The two phases A and B are heated to approximately 70-75° C.
2. Phase B is incorporated into phase A under stirring. Stirring for 15 to 20 minutes is then necessary in order to hydrate the phospholipids.
3. The mixture is homogenized at 5,000 rpm (Silverson or Ultra Turrax device) for 5 minutes.
4. Phases C and D are introduced into this mixture under stirring and the product thus formed is well mixed until homogenized.
5. The product is left to cool down under gentle stirring.
6. At approximately 35° C., phases E and F are introduced.
7. The mixture is homogenized at 1,500 rpm for 1 minute in order to avoid recrystallization during a long storage period.

Characteristics of the Composition:

| | | Viscosity | |
|---|---|---|---|
| | | in mPa · s | |
| Characteristics | appearance | (6 rpm, 3 min) | pH |
| Anti-ageing | white colour, | 50,000-60,000 | 5.2-5.7 |
| moisturizing | thick, homogeneous, | | |
| cream | perfumed | | |

Anti-Ageing Moisturizing and Softening Lotion:

This composition has an acid pH.

Composition in Percentages:

| Phase | Ingredient | INCI (Suppliers) | in % |
|---|---|---|---|
| A | Demineralized water | | qsf 100% |
| | EDTA, 4Na | Tetrasodium EDTA | 0.10 |
| | | (Lambert Rivière) | |
| | Trimethylglycine | Betaine(Finfeeds/LMC) | 3.00 |
| | Glycerin | Glycerin (Interchimie) | 2.00 |
| | Elestab CPN | Chlorphenesin | 0.10 |
| | | (Lab. Serobiologiques) | |
| B | Grapeseed oil | grapeseed oil | 5.00 |
| | | (Vitis vinifera) | |
| | | (IES) | |
| | Phytosqualane | squalane (Sophim) | 5.00 |
| | Cetiol SB45 | Butyrospermum parkii(Cognis) | 2.00 |
| | Vitamin E acetate | tocopheryl acetate | 0.10 |
| | | (Laserson) | |
| | Phenonip | phenoxyethanol + paraben | 0.50 |
| | | esters (Clariant) | |
| | Lucagel | | 2.50 |
| C | Isocell ®life | Water + lecithin + | 3.00 |
| | | Glycerol + | |
| | | butylene glycol + | |
| | | acrylates/sodium | |
| | | styrene copolymer | |
| | | (Lucas Meyer Cosmetics) | |
| D | Demineralized water | Water | 2.00 |
| | Laminactine ® | Glycerin + water + | 2.00 |
| | | lysolecithin + | |
| | | Perilla frutescens | |
| | | seed oil (Lucas Meyer | |
| | | Cosmetics) | |
| E | Citric acid 15% | Water + citric acid | 6.67 |
| F | FDC Blue 1 | CI 42090 (LCW) | qs |
| | FDC yellow 5 | CI 19 140 (LCW) | qs |
| G | C-3847 | Fragrance (Vanessence) | 0.20 |

Operating Method:
1. The two phases A and B are heated to approximately 60° C.
2. Phase A is incorporated into phase B under stirring. The mixture is mixed until the cream is thick and homogeneous.
3. The product is left to cool down.
4. At 40° C. phases C and D are introduced and the mixture is mixed until homogenized.
5. Phase E is then introduced and the mixture homogenized.
6. At approximately 35° C., phase F is introduced and the mixture is mixed until a homogeneous product is obtained.

Characteristics of the Composition:

| | | Viscosity in mPa · s | |
|---|---|---|---|
| Characteristics | appearance | (60 rpm, 30 s) | pH |
| Anti-ageing | Grey colour, | 4,000-4,500 | 3.0-3.5 |
| moisturizing | fluid, glossy, | | |
| and softening | homogeneous, | | |
| cream | perfumed | | |

Anti-Ageing Moisturizing Mineral Cream:

This cream is particularly rich in mineral salts.

Composition in Percentages:

| Phase | Ingredient | INCI (Suppliers) | in % |
|---|---|---|---|
| A | Demineralized water | | qsf 100% |
| | Elastab CPN | Chlorphenesin (Lab. | 0.10 |
| | | Serobiologiques) | |
| B | Alphaflow 30 | Hydrogenated polyisobutene | 5.00 |
| | | (Creations Couleurs) | |
| | Phytosqualane | Squalane (Sophim) | 2.50 |
| | Crodamol W Dist. | stearyl heptanoate (Croda) | 2.50 |
| | Cetiol SB 45 | Butyrospermum parkii | 3.00 |
| | | (Cognis) | |
| | Vitamin E acetate | Tocopheryl acetate | 0.10 |
| | | (Laserson) | |
| | Phenonip | Phenoxyethanol + | 0.50 |
| | | paraben esters | |
| | | (Clariant) | |
| | DC 345 | cyclopentasiloxane | 3.00 |
| | | (Dow Corning) | |
| | Lucagel | | 2.50 |
| C | Mamaku V. E | Water + cyatheaceae extract | 2.00 |
| | | (Lucas Meyer Cosmetics) | |
| D | Laminactine ® | Glycerin + water + | 2.00 |
| | | lysolecithin + | |
| | | Perilla frutescens seed | |
| | | oil (Lucas Meyer Cosmetics) | |
| E | FDC RED 4 | CI 14700 (LCW) | qs |
| | FDC YELLOW 5 | CI 19140 (LCW) | qs |
| F | C-3847 | Fragrance (Vanessence) | 0.30 |

Operating Method:
1. The two phases A and B are heated to approximately 60° C.
2. Phase A is incorporated into phase B under stirring. The mixture is mixed until the cream is thick and homogeneous.
3. The product is left to cool down under stirring.
4. At 40° C., phases C and D are introduced and the mixture is mixed until homogenized.
5. At approximately 35° C. phases C, D, E and F are introduced.

Characteristics of the Composition:

| | | Viscosity in mPa · s | |
|---|---|---|---|
| characteristics | appearance | (30 rpm, 30 s) | pH |
| Anti-ageing | Salmon colour, | 15,000-20,000 | 6.0-6.5 |
| moisturizing | soft, glossy | | |
| mineral cream | homogeneous, | | |
| | perfumed | | |

Moisturizing and Refreshing Cream for Sensitive Skins:

This composition is used cold.

Composition in Percentages:

| Phase | Ingredient | INCI (Suppliers) | in % |
|---|---|---|---|
| A | Demineralized water | | qsf 100% |
| | EDTA, 4Na | (Lambert Rivière) | 0.10 |
| | Glycerin | (Interchimie) | 3.00 |
| | Trimethyl glycine | Betaine (Finfeeds/LMC) | 2.00 |
| B | Lucagel | | 2.50 |
| | phenonip | phenoxyethanol paraben | 1.00 |
| | | esters (Clariant) | |
| | phytosqualane | squalane (Sophim) | 4.00 |
| | DC 345 | cyclopentasiloxane | 4.00 |
| | | (Dow Corning) | |
| | Sweet almond oil | Prunus amygdalus dulcis | 4.00 |
| | | (IES) | |
| | Dub Inin | isononyl isonoanoate (Dubois) | 4.00 |
| | DC 200.5 CS | dimethicone (Dow Corning) | 4.00 |
| | Tocopherol | tocopherol (Laserson) | 0.10 |
| C | Isocell ®Life | water + lecithin + | 2.00 |
| | | glycerol + | |
| | | butylene glycol + | |
| | | acrylates/sodium styrene | |
| | | copolymer (Lucas Meyer | |
| | | Cosmetics) | |
| D | Suprem' ®Plum | water + plum extract | 2.00 |
| | | (Prunus domestica) + | |
| | | hydrogenated lecithin + | |
| | | polyglyceryl-3 | |
| | | diisostearate + | |
| | | Glycerin + glyceride | |
| | | stearate (Lucas Meyer | |
| | | Cosmetics) | |
| E | C-3810 | Fragrance (Vanessence) | 0.20 |

Operating Method:
1. The two phases A and B are mixed at ambient temperature under gentle stirring.
2. The product is mixed until the cream is thick and homogeneous.
3. Phases C, D and E are introduced successively and the mixture is mixed gently until homogenized.

Characteristics of the Composition:

| | | Viscosity in mPa · s | |
|---|---|---|---|
| Characteristics | appearance | (12 rpm, 3 mn) | pH |
| Anti-ageing | White colour, | 45,000-50,000 | 5.5-6.0 |
| moisturizing | soft, glossy, | | |
| mineral cream | homogeneous, | | |
| | perfumed | | |

Radiance-Revealing Cream

This composition is a cream containing particles and with an acid pH. It is particularly suitable for exfoliation.

Composition in Percentages:

| Phase | Ingredient | INCI (Suppliers) | in % |
|---|---|---|---|
| A | Demineralized | | qsf 100% |
| | water | | |
| | Glycerin | Glycerin (Interchimie) | 2.00 |
| | Elestab CPN | Chlorphenesin (Lab. | 0.10 |
| | | Serobiologiques) | |
| B | Cetiol SN | Cetearyl octanoate | 3.00 |
| | | (Cognis) | |
| | Isohexadecane | Isohexadecane (CCW) | 3.00 |
| | Lipex 203 | Mangifera indica | 3.00 |
| | | (Unipex) | |
| | DC 345 | Cyclopentasiloxane | 3.00 |
| | | (Dow Corning) | |
| | Cetyl palmitate | Cetyl Palmitate (Gattefosse) | 1.00 |
| | Salicylic acid | Salicylic acid (Saci -CFPA) | 1.00 |
| | phenonip | phenoxyethanol + paraben | 0.50 |
| | | esters (Clariant) | |
| | Lucagel | | 4.00 |
| C | Apricot kernel | Prunus armeniaca | 3.00 |
| | powder | (Alban Muller) | |
| D | Isocel ®L citrus | water + alcohol + | 2.00 |
| | | lecithin + Glycerin + | |
| | | Citrus limonum(Lucas | |
| | | Meyer Cosmetics) | |
| | Mamaku V. E | Water + cyatheaceae extract | 2.00 |
| | | (Lucas Meyer Cosmetics) | |
| E | FDC Yellow 5 | CI 19140 (LCW) | qs |
| | DC Red 33 | CI 17200 (LCW) | qs |
| F | C-3810 | Fragrance | 0.40 |

Operating Method:
1. The two phases A and B are heated to approximately 70° C.
2. Phase A is incorporated into phase B, under moderate stirring. The stirring is gradually increased in order to make it possible to obtain good homogenization (approximately 1500 to 2000 rpm). The stirring is reduced when the product becomes thick and homogeneous.
3. The product is left to cool down.
4. Phase C is introduced.
5. At approximately 35° C., phases D, E and F are introduced and the mixture is mixed slowly until completely homogenized.

Characteristics of the Composition:

| | | Viscosity in mPa · s | |
|---|---|---|---|
| Characteristics | appearance | (12 rpm, 3 mn) | pH |
| Radiance- | Peach-coloured | 40,000-50,000 | 3.0-3.5 |
| revealing cream | cream, thick and | | |
| | perfumed, with | | |
| | chestnut brown | | |
| | particles. | | |

Matifying Cream with Clay and Oils:

Composition in Percentages:

| Phase | Ingredient | INCI (Suppliers) | in % |
|---|---|---|---|
| A | Demineralized water | | qsf 100% |
| | Veegum HV | Magnesium Aluminium | 1.00 |
| | | Silicate (Vanderbilt) | |
| B | Glycerin | Glycerin (Interchimie) | 3.00 |
| | trimethyl glycine | betaine (Finfeeds/LMC) | 2.00 |
| | Elestab CPN | chlorphenesin (Lab. | 0.20 |
| | | Serobiologiques) | |
| | EDTA, 4Na | Tetrasodium EDTA (Lambert | 0.02 |
| | | Rivière) | |
| C | Biophilic ®S | C 12-16 alcohols + | 1.00 |
| | | lecithin + palmitic acid | |
| | | (Lucas Meyer Cosmetics) | |
| | Dub GVF | Glyceryl linoleate + | 2.00 |
| | | Glyceryl oleate + | |
| | | Glyceryl linoleate | |
| | | (Dubois) | |
| | Phytosqualane | squalane (Sophim) | 1.00 |
| | Vitamin E acetate | tocopheryl acetate | 0.50 |
| | | (Laserson) | |
| | Phenonip | phenoxyethanol + | 0.50 |
| | | paraben esters (Clariant) | |
| | DC 345 | cyclopentasiloxane | 5.00 |
| | | (Dow Corning) | |
| D | Dermosoft octiol | caprylyl glycol (DR | 0.50 |
| | | Straetmans/LMC) | |
| E | Ultra-ventilated clay | Kaolin (Argiletz) | 7.00 |
| F | Lucagel | | 1.50 |
| G | Demineralized water | | 15.00 |
| | Matipure ® | magnesium and | 3.00 |
| | | aluminium silicate + | |
| | | hydroxyethylcellulose + | |
| | | black cumin seed oil | |
| | | (Nigella sativa) + | |
| | | winter squash seed oil | |
| | | (Cucurbita pepo) + | |
| | | phospholipids | |
| | | (Lucas Meyer Cosmetics) | |

| | | |
|---|---|---|
| HC-3810 | Fragrance (Vanessence) | 0.35 |

Operating Method:
1. The two phases A and C are heated to approximately 70-75° C.
2. Phase B is incorporated into phase A.
3. Phase C is then introduced. The mixture is left under gentle stirring for 15 to 20 minutes in order to hydrate the phospholipids.
4. The mixture is then homogenized by vigorous stirring at 2,000 rpm (Silverson or ultra Turrax device) for 1 minute.
5. Phases D and E are introduced.
6. Phase F is introduced and the mixture obtained is stirred until a homogeneous product is obtained.
7. Phase G is then introduced and the mixture is left to cool down
8. At approximately 35° C., phase H is introduced.

Characteristics of the Composition:

| | | Viscosity in mPa · s | |
|---|---|---|---|
| Characteristics | appearance | (6 rpm, 3 mn) | pH |
| Mattifying cream | Off-white-coloured | >90,000 | 6.5-7.0 |
| with oils | cream (variable | | |
| | according to the | | |
| | type of clay), thick | | |
| | and perfumed. | | |

Lightening Cream:

This composition has a basic pH and contains electrolytes (MAP). Lucagel is moreover coupled with another emulsifying agent

Composition in Percentages:

| Phase | Ingredient | INCI (Suppliers) | in % |
|---|---|---|---|
| A | Demineralized water | | qsf 100% |
| | Butylene glycol | Butylene Glycol | 2.00 |
| | | (Interchimie) | |
| | Satiaxane CX91 | xanthan gum (Laserson) | 0.20 |
| | Na Citrate | sodium citrate | 0.30 |
| | | (Lambert Rivière) | |
| | Triethanolamine | (Laserson) | qsf pH 8 |
| B | Amisol ®Soft | behenic alcohol + | 5.00 |
| | | glyceryl stearate + | |
| | | phospholipids + | |
| | | soybean sterols | |
| | | (soya glycine) | |
| | | (Lucas Meyer Cosmetics) | |
| | Lanette 16 | cetyl alcohol (Cognis) | 4.00 |
| | glyceryl stearate | Glyceryl stearate | 1.50 |
| | | (Dubois) | |
| | Nipanox BHT | BHT (Clariant) | 0.05 |
| | Phenonip | phenoxyethanol + | 1.00 |
| | | paraben esters | |
| | | (Clariant) | |
| | Parsol MCX | ethylhexyl | 5.00 |
| | | methoxycinnamate | |
| | | (Givaudan) | |
| | DC 345 | cyclopentasiloxane | 6.00 |
| | | (Dow Corning) | |
| | Cetiol SN | cetearyl octanoate | 4.00 |
| | | (Cognis) | |
| | Bisabolol | | 0.50 |
| C | Lucagel | | 1.00 |
| D | Demineralized water | | 12.00 |
| | EDTA, 4NA | Tetrasodium EDTA | 0.20 |
| | | (Lambert Riviere) | |
| | MAP | magnesium ascorbyl | 3.00 |
| | | phosphate (Cosmotochem) | |
| E | Butylene glycol | Butylene Glycol | 2.00 |
| | | (Interchimie) | |
| | UV Titanium M212 | CI 77891 (Kemira) | 2.00 |
| F | C-3221/4 | Fragrance | 0.35 |
| | | (Vanessence) | |

Operating Method:
1. The two phases A and C are heated to approximately 70-75° C.
2. Phase B is incorporated into phase A under gentle stirring. The mixture is maintained under gentle stirring for 15 to 20 minutes in order to hydrate the phospholipids.
3. The mixture is then homogenized by vigorous stirring at 3,000 rpm for 3 minutes.
4. Phase D is introduced and the mixture is mixed until homogenized.
5. The product is left to cool down, under gentle stirring.
6. At 40° C. (when the viscosity reduces), phase D is added and the mixture is mixed until homogenized.
7. At 35° C., phases E and F are introduced and the mixture is mixed until homogenized.
8. Finally, the product is homogenized for 1 minute at 1,500 rpm in order to avoid recrystallization during a long storage period. The pH is adjusted if necessary.

Characteristics of the Composition:

| | | Viscosity | |
|---|---|---|---|
| | | in mPa · s | |
| Characteristics | appearance | (6 rpm, 3 mn) | pH |
| Lightening cream | White-coloured cream, | 70,000-80,000 | 8.0-8.5 |
| | thick, glossy | | |
| | and perfumed. | | |

Slimming Gel-Cream:

This gel is a slimming agent with caffeine (Isocell® slim) and sodium salts (sodium salicylate)

Composition in Percentages:

| Phase | Ingredient | INCI (Suppliers) | in % |
|---|---|---|---|
| A | Demineralized water | | qsf 100% |
| | EDTA, 4Na | Tetrasodium EDTA | 0.10 |
| | | (Lambert Rivière) | |
| | Glycerin | Glycerin (Interchimie) | 3.00 |
| | Elestab CPN | chlorphenesin (Lab. | 0.10 |
| | | Serobiologiques) | |
| | Satiaxane CX91 | xanthan gum (Laserson) | 0.20 |
| B | Cetiol SN | cetearyl octanoate | 5.00 |
| | | (Cognis) | |
| | DC 345 | cyclopentasiloxane | 5.00 |
| | | (Dow Corning) | |
| | Vitamin E acetate | tocopheryl acetate | 0.10 |
| | | (Laserson) | |
| | Phenonip | Phenoxyethanol + | 0.50 |
| | | paraben esters | |
| | | (Clariant) | |
| | Lucagel | | 5.00 |
| C | Demineralized water | | 15.00 |
| | Isocell ®slim | Caffeine + sodium | 5.00 |
| | | salicylate + lecithin + | |
| | | silica (Lucas Meyer | |
| | | Cosmetics) | |
| D | Mamaku V. E | water + cyatheaceae | 2.00 |
| | | extract (Lucas Meyer | |
| | | Cosmetics) | |
| E | C-3810 | Fragrance (Vanessence) | 0.20 |

Operating Method:
1. Phases A, B and C are heated separately at approximately 60-65° C. under gentle stirring until homogenized.
2. Phase A is introduced into phase B under stirring. The stirring is increased until a thick and homogeneous product is obtained.
3. Phase C is added under gentle stirring. The stirring is maintained until a homogeneous product is obtained.
4. The mixture is left to cool down.
5. Phases D and E are introduced and the mixture is maintained under stirring until a homogeneous mixture is obtained.

Characteristics of the Composition:

| | | Viscosity | |
|---|---|---|---|
| | | in mPa · s | |
| Characteristics | appearance | (60 rpm, 30 s) | pH |
| Lightening cream | Salmon-coloured | 4,000-5,000 | 5.5-6.0 |
| | gel-cream, fluid, | | |
| | glossy, soft | | |
| | and perfumed. | | |

Make-Up Foundation:

Composition in Percentages:

| Phase | Ingredient | INCI (Suppliers) | in % |
|---|---|---|---|
| A | Demineralized water | | qsf 100% |
| | EDTA, 4Na | Tetrasodium EDTA | 0.10 |
| | | (Lambert Rivière) | |
| | Dermosoft octiol | Capryl glycol | 0.50 |
| | | (Dr Straetmans/LMC) | |
| | Lucagel | | 5.00 |
| B | Phenonip | Phenoxyethanol + | 0.50 |
| | | paraben esters | |
| | | (Clariant) | |
| | Dub Inin | isononyl isonoanoate | 10.00 |
| | | (Dubois) | |
| | Melanin mimic | Titanium dioxide | 10.00 |
| | | CI77499, CI77491 | |
| | | CI77942, hydrogenated | |
| | | decene oligomers | |
| | | Apricot kernel oil | |
| | | PEG40 ester, cetearyl | |
| | | Glucoside, cetearyl alcohol, | |
| | | Polyhydroxystearic acid, | |
| | | alumina dimethicone (Granula) | |

Operating Method:
1. Phases A, B and C are heated separately at approximately 60-65° C. under gentle stirring until homogenized.
2. Phase B is introduced into phase A under stirring. Phase C is added under gentle stirring until a homogeneous mixture is obtained.

Conditioner: (Capillary Care without Surfactant)

Composition in Percentages:

| Phase | Ingredient | INCI (Suppliers) | in % |
|---|---|---|---|
| A | Demineralized | | qsf 100% |
| | water | | |
| | EDTA, 4Na | Tetrasodium EDTA | 0.20 |
| | | (Lambert Rivière) | |
| | Glycerin | (Interchimie) | 2.00 |
| B | DC 1401 | Dimethicone Copolyol | 8.00 |
| | | (Dow Corning) | |
| | Lucagel | | 3.00 |
| | Phenonip | Phenoxyethanol + | 0.50 |
| | | paraben esters | |
| | | (Clariant) | |
| C | Demineralized | | 10.00 |
| | water | | |
| | Leogard GP | Polyquaternium 10 | 0.20 |
| | | (Akzo Nobel) | |
| | EDTA, 4Na | Tetrasodium EDTA | 0.20 |
| | | (Lambert Rivière) | |
| D | Essential oil of | Orange Sweet Oil | 0.20 |
| | sweet orange | (Australian Botanical) | |
| E | Citric acid 15% | Water + citric | up to pH 5 |
| | | acid | |

Operating Method:
1. Phases A and B are heated separately at approximately 60-65° C. under gentle stirring until homogenized.
2. Phase A is introduced into phase B under stirring. Phase C is added under gentle stirring until a homogeneous mixture is obtained.
3. The mixture is left to cool down
4. At 35° C., phases D and E are introduced and the mixture is homogenized.

Sun-Care Gel-Cream:

Composition in Percentages:

| Phase | Ingredient | INCI (Suppliers) | in % |
|---|---|---|---|
| A | Parsol MCX | Ethylhexyl methoxycinnate | 7.50 |
| | | (Givaudan) | |
| | Parsol 1789 | Butyl methoxydibenzoylmethane | 2.00 |
| | | (Givaudan) | |
| | Eusolex 6300 | 4 methylbenzylidene | 2.50 |
| | | camphor (Merck) | |
| | Phenonip | Phenoxyethanol + | 0.50 |
| | | paraben esters (Clariant) | |
| | Dermosoft octiol | Capryl glycol | 0.50 |
| | | (DR Straetmans/LMC) | |
| | Vitamin E acetate | tocopheryl acetate | 1.00 |
| | | (Laserson) | |
| | DC 593 | dimethicone + | 1.00 |
| | | trimethylsiloxysilicate | |
| | | (Dow Corning) | |
| | Abil AV 1000 | phenyl trimethicone | 1.00 |
| | | (Degussa) | |
| | DC 345 | Cyclopentasiloxane | 5.00 |
| | | (Dow Corning) | |
| | Alphaflow 30 | Hydrogenated polyisobutene | 4.00 |
| | | (Creations Couleurs) | |
| | Cetiol SB 45 | Butyrospermum parkii | 1.50 |
| | | (Cognis) | |
| | Lucagel | | 3.50 |
| B | Demineralized water | | qsf 100% |
| | Glycerin | Glycerin (Interchimie) | 3.00 |
| | EDTA, 4Na | Tetrasodium EDTA (Univar) | 0.02 |
| | Elestab CPN | Chlorphenesin (Lab. | 0.20 |
| | | Serobiologiques) | |
| C | Z-COTE | CI-77497(Sun Smart) | 5.00 |
| D | C-3043/23 | Fragrance (Vanessence) | 0.35 |

Operating Method:
1. Phases A and B are heated separately at approximately 60-65° C. under gentle stirring until homogenized.
2. Phase A is introduced into phase B under stirring until a homogeneous mixture is obtained.
3. The mixture is left to cool down.
4. At 35° C., phases C and D are introduced and the mixture is homogenized.

Characteristics of the Composition:

| | | Viscosity | |
|---|---|---|---|
| | | in mPa · s | |
| Characteristics | appearance | (6 rpm., 3 mn) | pH |
| Lightening cream | White-coloured | 50,000-60,000 | 7.0-7.5 |
| | gel-cream, glossy, | | |
| | homogeneous | | |
| | and perfumed. | | |

Sun-Care Gel-Cream:

This composition is a physical and chemical sun-care product

Composition in Percentages:

| Phase | Ingredient | INCI (Suppliers) | in % |
|---|---|---|---|
| A | Demineralized | | qsf 100% |
| | water | | |
| | Lucagel | | 5.00 |
| | Phenonip | Phenoxyethanol + | 0.50 |
| | | paraben esters | |
| | | (Clariant) | |
| | Dermosoft | Caprylyl glycol | 0.50 |
| | octiol | (DR Straetmans/LMC) | |
| | EDTA, 4Na | Tetrasodium EDTA | 0.10 |
| | | (Lambert Rivière) | |
| B | Dub Inin | isononyl isonoanoate | 10.00 |
| | | (Dubois) | |
| | Granlux GAI-45 | Titanium dioxide, | 10.00 |
| | | isononyl isononanoate, | |
| | | Polyglyceryl 4 isostearate, | |
| | | cetym PEG/PPG 10/1, | |
| | | Dimethicone, hexyl laurate, | |
| | | aluminium stearate (Granula) | |

Operating Method:
1. Phase A is heated at approximately 60-65° C. under gentle stirring until homogenized.
2. Then it is left to cool down
3. At 35° C. phase A is introduced into phase B under stirring until a homogeneous mixture is obtained.

1. Gelling and/or thickening agent characterized in that it is formed from a combination of at least three constituents: a polyacrylamide and ammonium acrylate copolymer and/or anionic acrylic copolymer, phospholipids of plant origin, and a polyglyceryl acrylate.

2. Gelling and/or thickening agent according to claim 1, characterized in that it contains a copolymer concentration ranging from 20 to 50% of the total weight of the agent, a phospholipid concentration ranging from 6 to 40% and a polyglyceryl acylate concentration ranging from 5 to 40%.

3. Gelling and/or thickening agent according to claim 1 in which the copolymer is present at a concentration ranging from 50 to 80% in a dilute form in a hydrogenated polyisobutene containing from 40% to 60% of active material.

4. Gelling and/or thickening agent according to claim 1, in which the phospholipids are in the form of lecithins which are present in a concentration ranging from 10% to 40% of the total weight of the agent.

5. Gelling and/or thickening agent according to claim 4 in which the lecithins contain less than 40% oil and more than 60% phospholipids.

6. Gelling and/or thickening agent according to claim 1, in which the acrylic copolymer is a sodium acrylate/acryloyldimethyl taurate copolymer.

7. Gelling and/or thickening agent according to claim 1, in which the polyglyceryl acylate is a stearate, a distearate or a linoleate.

8. Gelling and/or thickening agent according to claim 7, in which the polyglyceryl acylate is a polyglyceryl-10 stearate.

9. Gelling and/or thickening agent according to claim 1, constituted by the three constituents: polyacrylamide and/or ammonium acrylate copolymer or anionic acrylic copolymers, phospholipids of plant origin and polyglyceryl acrylate, these three constituents completing the agent to 100% by weight.

10. Aqueous, fluid or gelled composition, intended to be applied to the skin, mucous membranes or teguments, characterized in that it contains, as active ingredient, at least one gelling and/or thickening agent according to claim 1, in combination or in a mixture with one or more excipients or vehicles appropriate for cosmetic or dermatological use.

11. Cosmetic or dermatological composition according to claim 10, wherein it is produced in the form of an oil-in-water emulsion, with or without additional emulsifying agent, said gelling and or thickening agent, in the form of a gel, being incorporated into the oil phase or into the aqueous phase of said compositions.

12. Cosmetic or dermatological composition according to claim 10 in which the average size of the particles in emulsion ranges from 1 to 11 μm.

13. Cosmetic or dermatological composition according to claim 10, in which the gelling and/or thickening agent is present in a concentration ranging from 0.05 to 10% of the total weight of the composition.

14. Cosmetic or dermatological composition according to claim 10, in which the gelling and/or thickening agent is present in a concentration ranging from 0.2 to 4%.

15. Cosmetic or dermatological composition according to claim 10, in which the vehicle is water or an aqueous vehicle, in a proportion ranging between 20 to 70%.

16. Use of the gelling and/or thickening agent according to claim 1, as stabilizing and/or thickening agent in an emulsion with a cosmetic or dermatological character.

17-18. (canceled)

19. A process for producing an oil-in-water emulsion comprising incorporating in the oil phase the gelling and/or thickening agent according to claim 1, and adding an aqueous phase to the latter.
